(19) United States
(12) Plant Patent Application Publication
(10) Pub. No.: US 2003/0106116 P1
(43) Pub. Date: Jun. 5, 2003

(54) Hybrid tea rose plant named 'Tantalizing Red'

(76) Inventor: James W. Coiner, La Verne, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/016,070

(22) Filed: Dec. 5, 2001

(51) Int. Cl. A01H 5/00

(52) U.S. Cl. PLT/138

(57) ABSTRACT

A new and distinct Hybrid Tea Rose plant variety with red flowers.

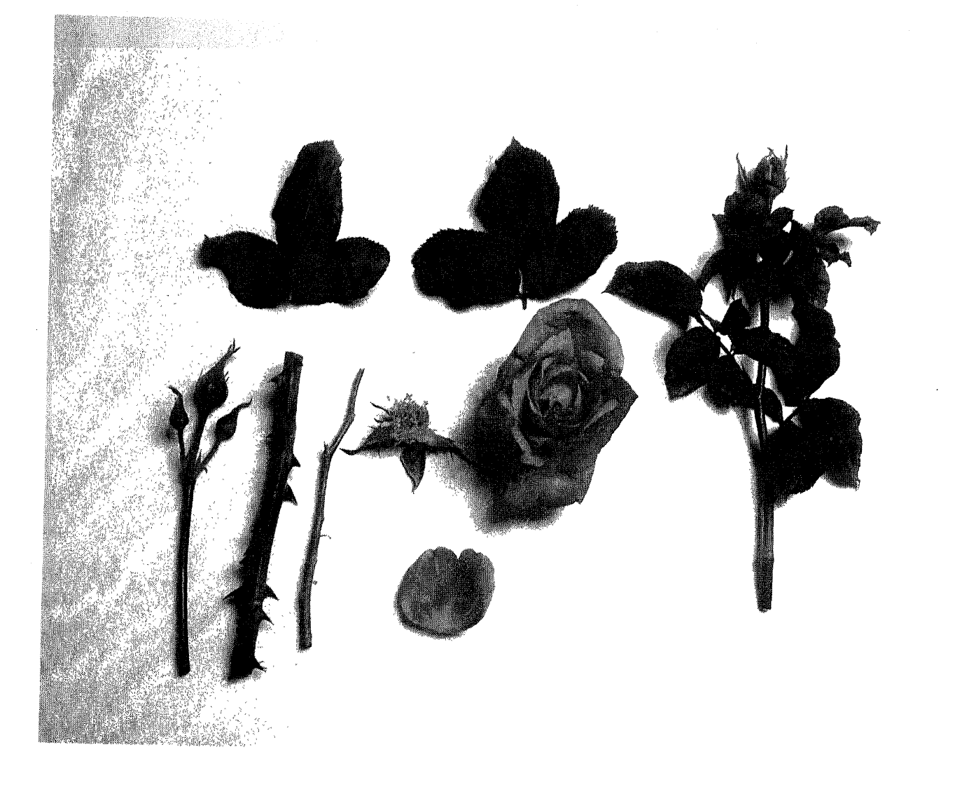

### BACKGROUND OF THE INVENTION

The present invention relates to a new and distinct variety of rose plant of the hybrid tea rose which was created by crossing as seed and pollen parents two (2) unknown rose varieties. The varietal denomination of the new variety is ‘TANTALIZING RED’.

### SUMMARY OF THE INVENTION

Among the novel characteristics possessed by this new variety which distinguishes it from other varieties of which I am aware, is its attractive red flowers.

Asexual reproduction by propagation by budding of the new variety as performed in Wasco, Calif. shows that the foregoing and other distinguishing characteristics come true to form and are established and transmitted through succeeding propagations.

### BRIEF DESCRIPTION OF ILLUSTRATION

The accompanying illustration shows typical specimens of the flowers of the new variety depicted in color as nearly true as it is reasonably possible to make the same in a color illustration of this character.

### DESCRIPTION OF THE NEW VARIETY

The following is a detailed description of 2-year budded plants of the new variety , with color terminology in accordance with The Royal Horticultural Society Colour Chart (RHSCC). The terminology used in color description herein refers to plate numbers in the aforementioned color chart. Phenotypic expression may vary with light availability, environmental and cultural conditions.

The following observations are made of specimens grown outdoors in Wasco, Calif., during November.

### PLANT

Form: Upright

Growth: Vigorous, non-uniform

Size: Height about 6 feet; width about 3 feet

Foliage

Size.—Length about 2 ½ inches; width about 1 ¾ inches.

Quantity.—About 7

Color.—New foliage: Upper side near 146A; under side near 182B Mature Upper side near 147A; under foliage: side near 147C

Shape.—Elliptic.

Texture.—Upper side: Smooth Under side: Matte

Edge.—Serrated.

Serration.—Slight.

Petioles: Top surface color near 144B;

Under side near 144A

Stipules: Normal

Auricle: Alan shaped

Rachis: Smooth

Variegation: Slight

Veination: None observed

### WOOD

New shoots

Bark.—Smooth.

Wood.—Near 60A

Old wood

Bark.—Smooth.

Wood.—Near 146B.

Flowering stem: Length about 6 inches

Winter Hardiness: Completely winter hardy

Resistance to Disease: Average

Temperature range: Between 16° and 120° F.

Internode spacing: Regular

Preferred growing conditions: Full sun

Growth habit: Upright

Leaves per stem: About 7

### FLOWER/INFLORESCENCE

Blooming Habit: Recurrent

Bud.—About 1 3/16 inch Form: High centered classic rose form Color: Before sepals divide color near 60A; when sepals first divide color near 60C; when petals begin to unfurl color near 63B

Sepals.—Pointed on ends; regular appearance. Size: Length in relation to bud, about 1 ⅜ inches Color Topside near 158B; bottom near 144C

Peduncle.—Length: About 1 ¾ inches Strength: Firm Color: Near 59C.

Flower

Size.—About 5 inches.

Borne.—Singly.

Form.—High centered.

Petalage: About 38

Petaloids in center.—Few.

Persistence.—Strong.

Fragrance: Slight

Lasting Quality: About 3 weeks on plant; about 6 days after cutting

Color

During first 2 days.—Inner petals at flower center topside near 65B; bottom near 65C; outer petals topside near 65B, bottom near 65C.

Base of petals (Point of attachment): Near 158B

General tonality from a distance: Near 65B

Color Change: As bloom ages, fades slight to near 65C; after 3 or more days, about the same

General tonality from a distance: Dark red even after 3 or more days

Petals

Texture.—Soft.

Shape.—Wider than long.

Arrangement.—Normal.

Appearance.—Satiny.

### REPRODUCTIVE ORGANS

Arrangement of stamens, Regularly arranged around

Filaments and Anthers: styles

Color: Filaments near 162B; Anthers near 158B

Styles: Thin

Stigmas: Color near 158D

Hips/Seed Vessels/Ovaries

Shape.—Round.

Size.—About ½ inch.

Color.—Near 144A.

Seeds: No seeds observed

I claim:

1. A new and distinct Hybrid Tea Rose plant of the variety substantially as shown and described.
